(19) United States
(12) Patent Application Publication
(10) Pub. No.: US 2025/0295339 A1
(43) Pub. Date: Sep. 25, 2025

(54) APPARATUS, SYSTEM AND METHOD FOR MONITORING PATIENTS FOR INTERNAL INFECTION

(71) Applicants: Pei-Lun Chen, Philadelphia, PA (US); Sharvan Ranganathan, Monroe, NY (US); Iris Layadi, Atlanta, GA (US)

(72) Inventors: Pei-Lun Chen, Philadelphia, PA (US); Sharvan Ranganathan, Monroe, NY (US); Iris Layadi, Atlanta, GA (US)

(21) Appl. No.: 18/979,041

(22) Filed: Dec. 12, 2024

Related U.S. Application Data
(60) Provisional application No. 63/568,625, filed on Mar. 22, 2024.

(51) Int. Cl. A61B 5/20; A61B 5/00; A61B 5/145; A61B 10/00

(52) U.S. Cl. CPC A61B 5/207; A61B 5/14507; A61B 5/742; A61B 10/007; A61B 2010/0006; A61B 2560/0462

(57) ABSTRACT

An apparatus for monitoring patients for internal infection includes a urine reservoir in fluid communication with urine flowing from the catheter to the urine bag. A dispensing unit is capable of moving a continuous feed of test strips through the urine reservoir such that the test strips come in contact with urine in the urine reservoir. The test strips comprise color-changing urinary tract infection test strips. The test strips contacting the urine define used test strips and test strips not having yet contacting the urine define unused test strips. A sensing module has an optical path in communication with a newest one of the used test strips to optically sense a color thereof. Communication of the color to a communicative processor enables recognition of the urinary tract infection. The dispensing unit is capable of intermittently moving unused test strips into the urine reservoir.

### CROSS-REFERENCE TO RELATED APPLICATIONS

I hereby claim the benefit under 35 U.S.C. Section 119 (e) of U.S. Provisional application 63/568,625 filed on Mar. 22, 2024.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure is related to healthcare systems and methods for monitoring patients for internal infections.

(2) Description of Related Art including information disclosed under 37 CFR 1.97 and 1.98.

The lack of early detection and monitoring tools for catheter-associated urinary tract infections (CAUTIs) highlights an evident need for a clinical solution. In the United States alone, CAUTIs represent a significant healthcare challenge, affecting over 560,000 patients annually and constituting more than 40% of hospital-acquired infections. As the predominant healthcare acquired disease, CAUTI leads to increased patient morbidity, length of hospital stays, and associated healthcare costs, making the early and accurate detection of CAUTIs essential.

The over-reliance of current diagnostic procedures on symptom monitoring often causes diagnostic and treatment delays, subsequently increasing the risk of developing secondary complications. This issue is exacerbated in resource-limited care facilities and amongst the elderly patient population, who frequently present with ambiguous symptoms.

Currently, CAUTI is detected definitively via urine cultures and urinalysis tests, which are typically reactive and take days to produce results. This lag in detection significantly compromises patient outcomes, especially when executed after symptom onset. Despite advances in this specialty area, a fully integrated, automated, and real- time monitoring solution has been absent from the market.

Thus, the known art lacks devices that cater to early CAUTI detection as a supplement to definitive laboratory urinalysis and urine cultures. The current standard of care is therefore too heavily dependent on clinical monitoring, which is prone to diagnostic delays and the challenges associated with over and under treatment.

The foregoing disadvantages lead to increased patient discomfort and mortality, amplified healthcare expenditures, and extended hospital stays, presenting a significant burden on the healthcare system. As a result, the need exists for a continuous monitoring, point-of-care testing (POCT) apparatus, system and method to provide rapid onsite evaluation of early CAUTI indicators, facilitating preemptive diagnosis and intervention.

### BRIEF SUMMARY OF THE INVENTION

An embodiment of the disclosure meets the needs presented above by generally comprising an apparatus for monitoring patients for internal infection includes a urine reservoir in fluid communication with urine flowing from the catheter to the urine bag. A dispensing unit is capable of moving a continuous feed of test strips through the urine reservoir such that the test strips come in contact with urine in the urine reservoir. The test strips comprise color-changing urinary tract infection test strips. The test strips contacting the urine define used test strips and test strips not having yet contacting the urine define unused test strips. A sensing module has an optical path in communication with a newest one of the used test strips to optically sense a color thereof. Communication of the color to a communicative processor enables recognition of the urinary tract infection. The dispensing unit is capable of intermittently moving unused test strips into the urine reservoir.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1A is a front view of a display screen of an apparatus, system and method for monitoring patients for internal infection according to an embodiment of the disclosure.

FIG. 1A is a front view of a display screen of an apparatus, system and method for monitoring patients for internal infection according to an embodiment of the disclosure.

FIG. 1B is a front view of a display screen of an embodiment of the disclosure.

FIG. 1C is a front view of a display screen of an embodiment of the disclosure.

FIG. 1D is a front view of a display screen of an embodiment of the disclosure.

FIG. 2 rear view of a portion of a dispending unit of an embodiment of the disclosure.

FIG. 3 is a perspective view of a connection module of an embodiment of the disclosure.

FIG. 4A is a rear perspective view of an embodiment of the disclosure.

FIG. 4B is a rear perspective view of an embodiment of the disclosure.

FIG. 4C is a rear perspective view of a portion of dispensing unit of an embodiment of the disclosure.

FIG. 4D is a top perspective view of a test strip of an embodiment of the disclosure.

FIG. 5 is a schematic view of an embodiment of the disclosure.

FIG. 6A is a front perspective view of an embodiment of the disclosure.

FIG. 6B is a front perspective view of an embodiment of the disclosure.

FIG. 7 is a front view of an embodiment of the disclosure.

FIG. 8 is a cross-sectional view of an embodiment of the disclosure taken along line 8-8 of FIG. 6B.

FIG. 9 is a in-use view of an embodiment of the disclosure.

### DETAILED DESCRIPTION OF THE INVENTION

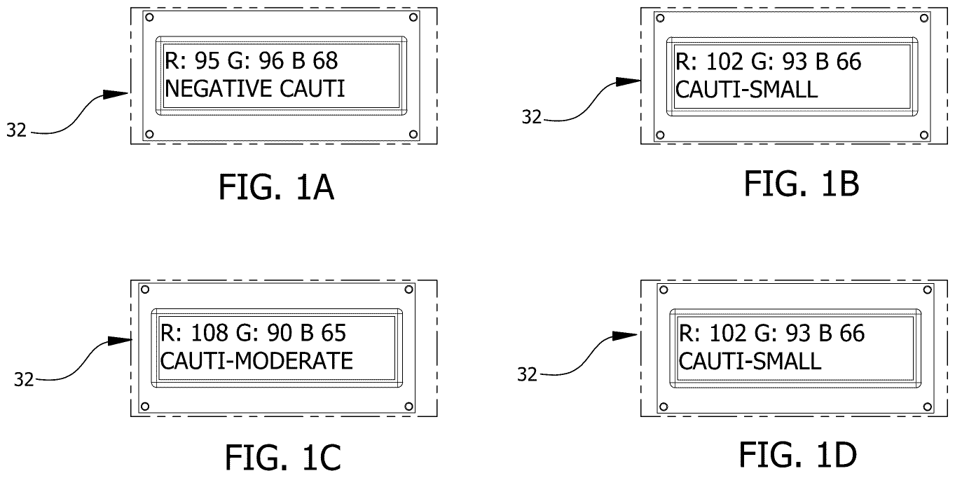

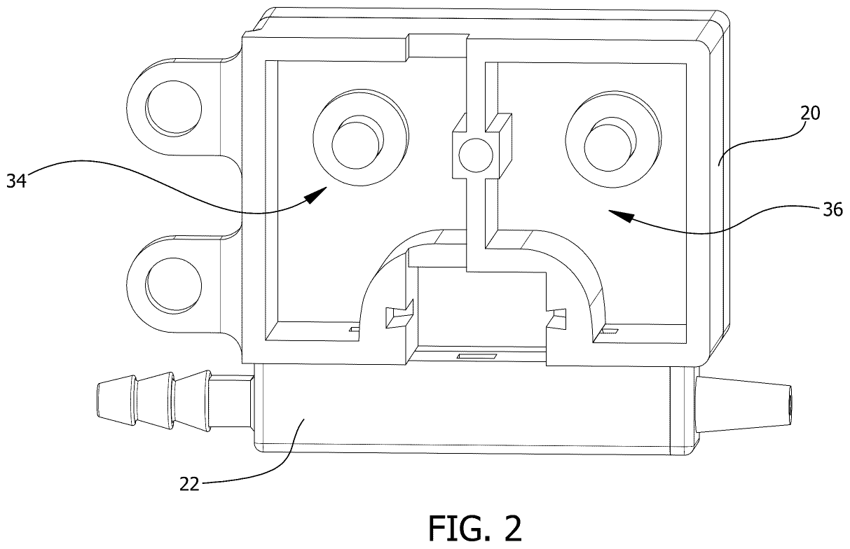

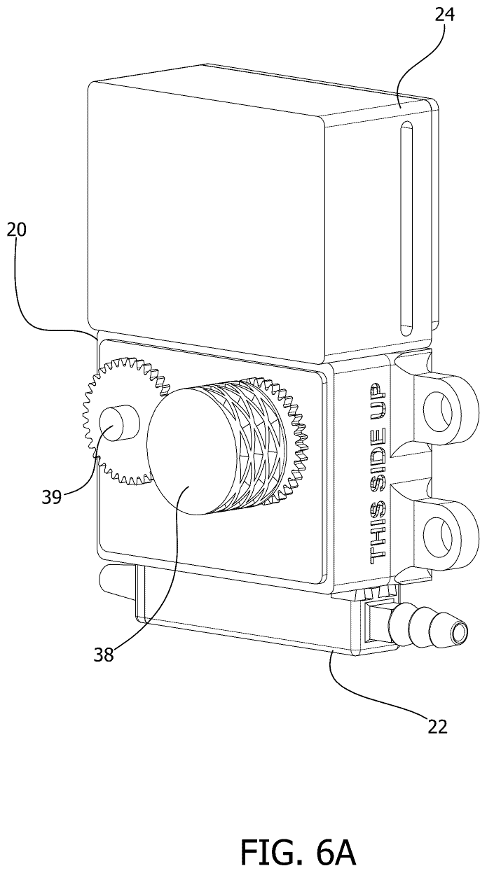

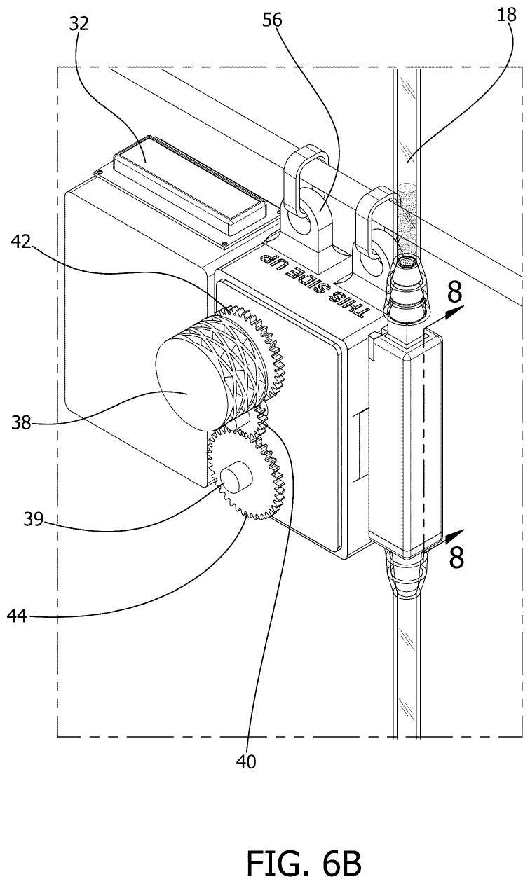

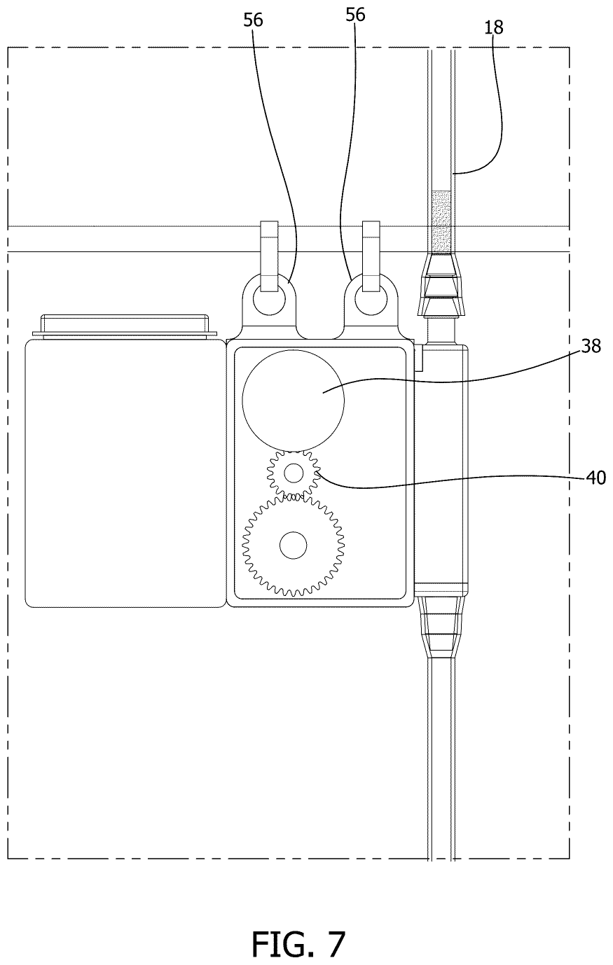

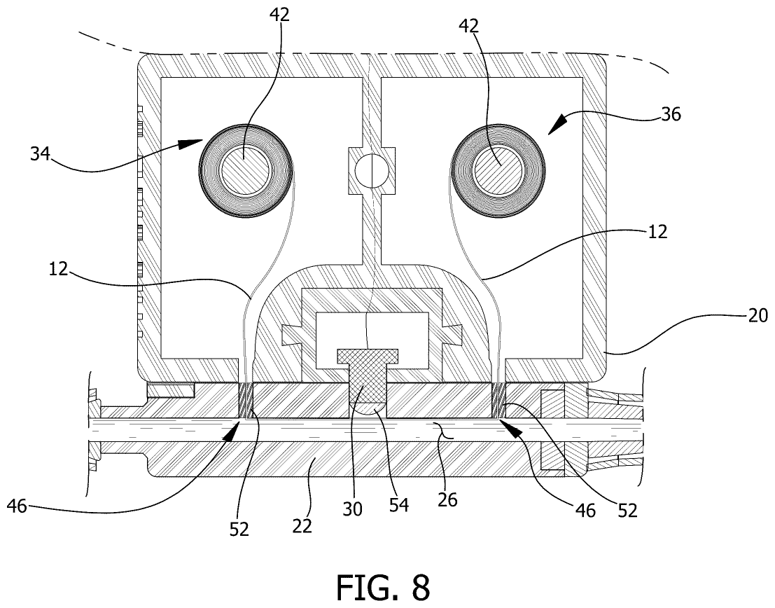

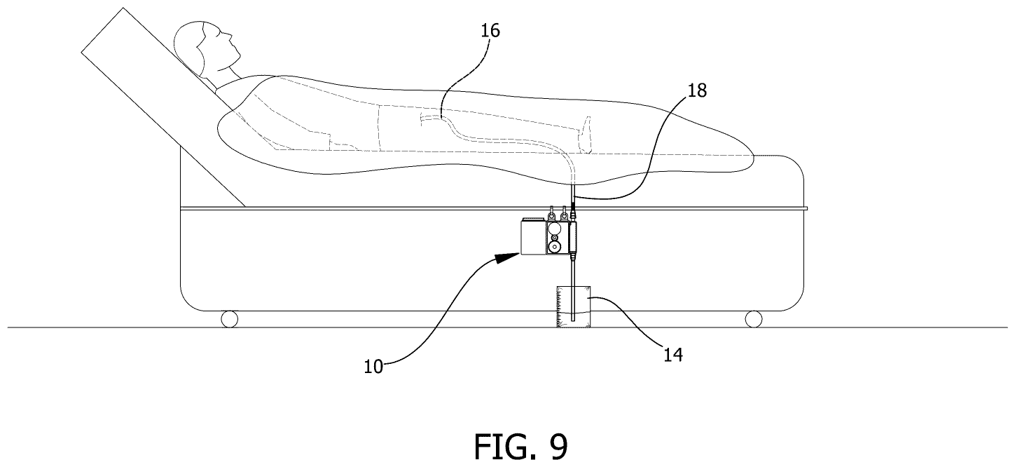

With reference now to the drawings, and in particular to FIGS. 1 through 9 thereof, a new infection monitoring device embodying the principles and concepts of an embodiment of the disclosure and generally designated by the reference numeral 10 will be described.

As best illustrated in FIGS. 1 through 9, to facilitate timely detection of CAUTI, the apparatus, system and method for monitoring patients for internal infection 10 generally comprises embodiments that integrate with existing medical workflows without compromising standard urine testing procedures. The embodiments provide immediate or near-immediate visual signal upon detection of CAUTI indicators in the urine. The embodiments thereby reduce the average hospital length of stay due to CAUTIs, and exhibit higher specificity and sensitivity rates compared to existing at-home UTI testing methods.

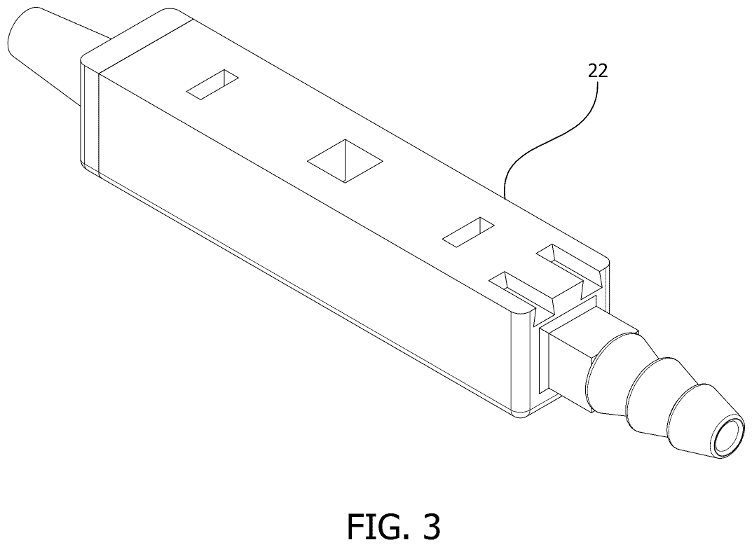

The connection module 22, as shown in FIG. 3, may include the mechanical capabilities to connect to existing foley catheter tubing and/or a urine bag 14. The connection module 22 includes a connection to the UTI test strip dispenser, and allows the test strip 12 to be fed in and out of the lumen created by the connection module 22. More specifically, the connection module 22 connects and is in the flow between the catheter 16 and the urine bag 18 and includes the integrated UTI test strip 12 dispenser/hosing. Urine 26 flows from the catheter 16 into the connector module 22, where it contacts the test strip 12 positioned within the connector module's 22 flow path. A precision gear system within the dispenser 20 may facilitate the movement of the test strip 12 into the flow of urine 26.

This connection module 22 may be universally compatible with various catheter and urine bag systems, eliminating the need for specialized manufacturing processes and thus reducing production costs. The connection module 22 may be positioned above the urine bag 18 to shorten the length of the urine bag 18 tubing, which aids in supporting both the urine bag 18 and the connection module 22. The top of the connection module 22 may include a three-tiered adapter for secure attachment to the drainage tubing 18, designed to prevent leaks. The bottom of the connection module 22 may comprise a streamlined design to ensure a direct flow into the urine bag 18, preventing backflow and kinking along the drainage tubing pathway.

The optical sensor 30, such as is illustrated in the exemplary embodiment of FIG. 3, may be housed within a casing attached to the test strip 12 dispenser 20. The optical sensor 30 detects and allows for analysis of RGB colors on the UTI test strip reagent pads 28. This connection to suitable analysis capabilities may be wired or wireless, and/or, in whole or in part, the analysis capability may be part of the optical sensor 30, such as resident in its firmware. Further, the optical sensor 30 and/or the analysis capabilities may be communicative with the display 32, such as that shown in FIGS. 1A-D.

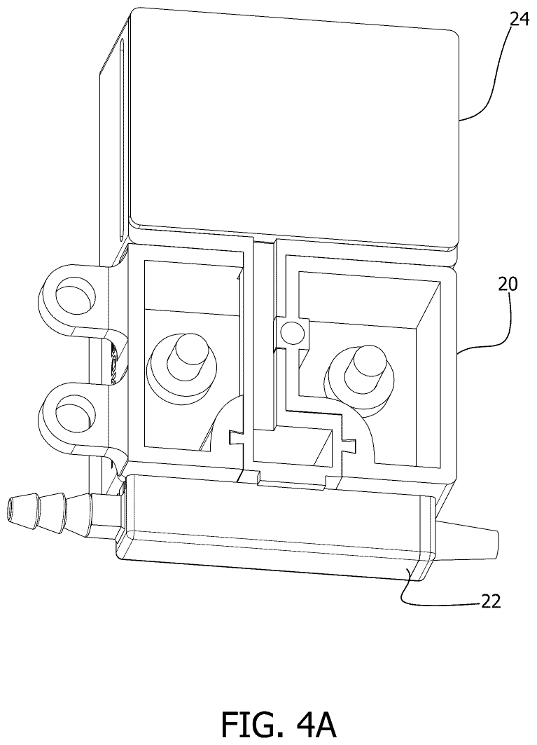

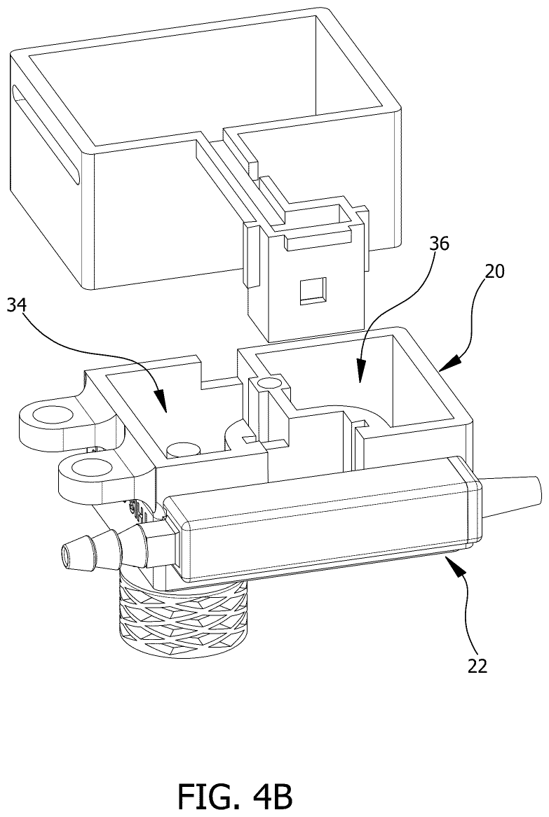

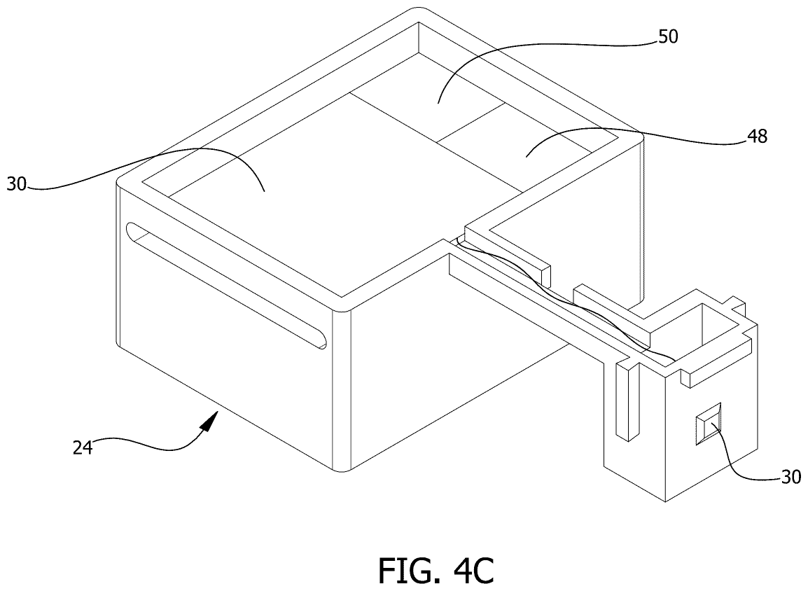

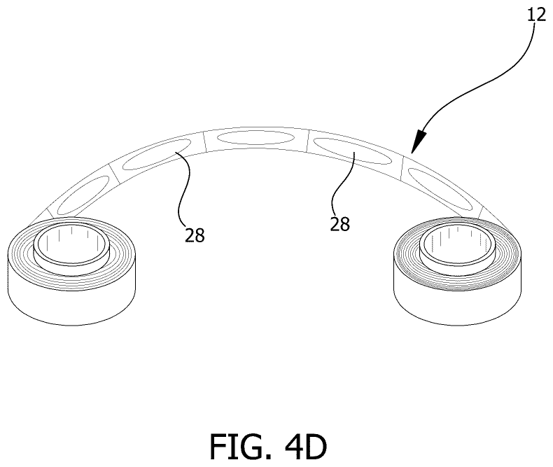

The full system 10, with the connection module 22 positioned to pass urine 26 onto the test strip 12, and expose the thereafter activated test strip to the optical sensor 30 staged so as to view a test strip 12 section once it is exposed to urine 26 in the connection module 22, is shown in FIG. 4A. Of note, the test strip 12 may move through the dispenser 20 from left to right as illustrated. That is, the clean test strip 12 is within the left, i.e. first 34, chamber, is exposed to the urine 26 and is sensed, and then moves to the right, i.e. second 36, chamber. Similarly, FIG. 4B illustrates the full unit 10, but with the top cover removed so as to more clearly illustrate the optical sensor 30 “looking” at the test strip 12 as the test strip 12 is exposed to the urine 26 flowing in the connection module.

FIG. 4B provides particular clarity in that it shows the sensor 30 housing open, and illustrates the view path from the sensor 30 to the test strip 12. The test strip 12 would be extending between the slits 46 on either side of the connection module 22 at the end of the view path, and the window that abuts the connection module 22 allows for entry of the urine 26 through the window and onto the test strip 26. Once the test strip 12 is wetted, it will change colors, which will be “seen” by the optical sensor 30 as it views along the view path. This is made more clearly evident in the isometric view of FIG. 4C.

More particularly in relation to the optical sensor 30, white light interacts with the urine 26, the sample selectively absorbs or reflects specific wavelengths of light contingent upon its inherent color characteristics. The sensor employs photodetectors sensitive to diverse wavelengths, capturing the spectral information of the light that has interacted with the urine sample.

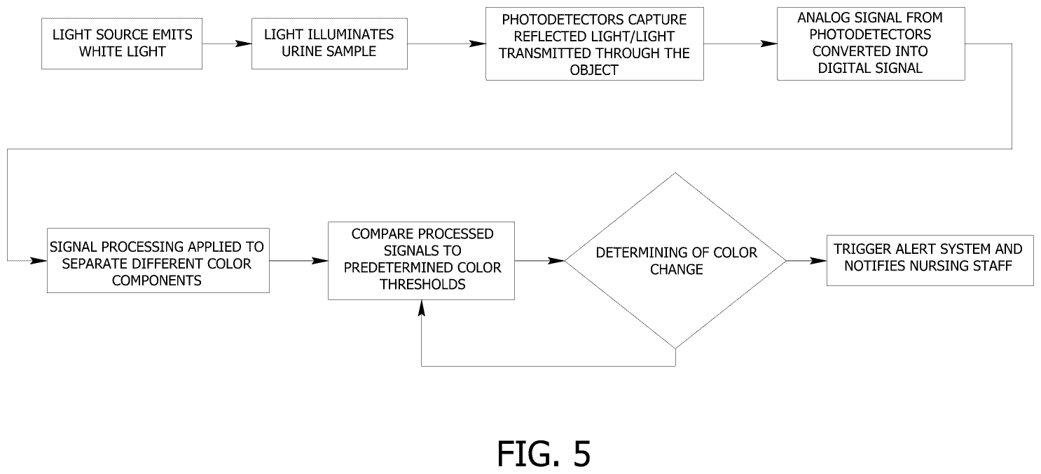

Subsequently, an analog-to-digital conversion process may translate this optical signal into a digital format for further analysis. Signal processing algorithms are then employed to extract and discern the spectral components representative of distinct colors within the urine sample. These spectral features are subsequently cross-referenced against predetermined color standards or thresholds stored in a computing memory. In the event of a substantial deviation from the established color reference values, the sensor registers a color change within the urine sample and generates an output or alert signal. The aforementioned process is further illustrated in relation to the flowchart in FIG. 5. In relation specifically to the optical sensor 30 and by way of non-limiting example, the optical sensor may be a TCS34725 optical sensor. An exemplary microcontroller, or processor 48, utilized for an optical sensor setup may be the Arduino Nano.

Needless to say, the disclosed system and method may additionally include other elements. By way of example, the measuring unit may include power, such as via a power feed or a battery 50. Further included may be a communications card, such as for wired or wireless communication into and out from the system 10. This communication may occur to a processor, which may itself communicate with an app, such as on a computer or a mobile device.

The disclosed solution thus directly integrates a test strip dispenser 20 with an optical sensor 30, enabling continuous, real-time surveillance of patients' urine status and substantially narrowing the delay between infection onset and detection. The system 10 differs from traditional urine cultures and symptomatic reporting by providing proactive, uninterrupted monitoring. Employing pre-validated FDA-approved test strips ensures reliability, while the optical sensor 30 employs precise RGB analysis to analyze color changes that may occur on the test strips' reagent pads, indicating the presence and specific level of leukocytes, enhancing diagnostic efficiency and accuracy.

Of additional note, the housing of the dispenser 20 as well as the optical sensing unit 24 may be made from PLA, which is robust and withstands external trauma and chemical processing needed for sterilization. The optical sensor 30 may be encased within a waterproof housing and may include a transparent polyethylene barrier between the sensor and the view path. The connection module 22 may be fabricated using PET-G, and may also be printed, together with the other components and sub-components, via additive manufacturing for ease of assembly. The connection module may feature universal connectors to ensure compatibility with a range of Foley catheter and drainage tubing brands and sizes.

Thereby, the disclosure includes UTI test strips 12 that can be fed via continuously from a novel dispensing design into a connection module 22, where it will become saturated with urine 26. The optical sensor component 24 remains the same as our initial design, but is located across from the opening on the connection module 22 that exposes the reagent pads 28 on the test strip 12, so that any color change will be captured and signaled to the nursing staff. The disclosed apparatus, system and method thereby consistently monitors urinary biomarkers (leukocytes) indicative of infection.

In sum, the disclosed system and method provides a manual or automated test strip dispenser for the continuous monitoring of patients' urinary status. Included may be a rotary test strip dispenser, a catheter and drainage bag-compatible connection module, and an optical sensor that executes RGB analysis. The device may operate within the existing infrastructure of urinary catheterization in clinical settings.

The embodiments may be used in hospital environments, long-term care facilities, and medical clinics where urinary catheterization is a standard practice, by way of non-limiting example. The embodiments enable the preemptive detection of CAUTIs, thus mitigating patient risk and reducing the economic burden associated with late-stage infection management. The embodiments provide a complementary detection and monitoring tool to support standard diagnostic procedures, including urinalyses and urine cultures.

In the foregoing detailed description, it may be that various features are grouped together in individual embodiments for the purpose of brevity in the disclosure. This method of disclosure is not to be interpreted as reflecting an intention that any subsequently claimed embodiments require more features than are expressly recited.

Further, the descriptions of the disclosure are provided to enable any person skilled in the art to make or use the disclosed embodiments. Various modifications to the disclosure will be readily apparent to those skilled in the art, and the generic principles defined herein may be applied to other variations without departing from the spirit or scope of the disclosure. Thus, the disclosure is not intended to be limited to the examples and designs described herein, but rather is to be accorded the widest scope consistent with the principles and novel features claimed as follows.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A unitary tract infection monitoring assembly configured to continuously monitor urine flowing from a catheter to a urine bag, said assembly including:
a urine reservoir in fluid communication with urine flowing from the catheter to the urine bag;
a dispensing unit capable of moving a continuous feed of test strips through the urine reservoir such that the test strips come in contact with urine in the urine reservoir, the test strips comprising color-changing urinary tract infection test strips, wherein test strips having come in contact with the urine defining used test strips and test strips not having yet come in contact with the urine defining unused test strips;
a sensing module having an optical path in communication with a newest one of the used test strips to optically sense a color thereof, wherein communication of the color to a communicative processor enables recognition of the urinary tract infection; and
wherein the dispensing unit is capable of intermittently moving unused test strips into the urine reservoir.

2. The urinary tract infection monitoring assembly according to claim 1, wherein the dispensing unit comprises two compartments, a first compartment holding the unused test strips and a second compartment receiving the used test strips.

3. The urinary tract infection monitoring assembly according to claim 2, further including at least two gears wherein the first compartment has one of the gears therein and the second compartment has one of the gears therein, the at least two gears engaging the continuous feed of test strips and urging the continuous feed of test strips from the first compartment and into the second compartment.

4. The urinary tract infection monitoring assembly according to claim 1, further including a connection module capable of receiving a urine feed from a patient and passing the urine feed to a urine bag, the connection module being in fluid communication with the urine reservoir to pass urine from the urine feed to the urine reservoir.

5. The urinary tract infection monitoring assembly according to claim 1, further including a display being electrically coupled to said communicative processor, sad display providing a visual reading of a urinary tract infection test result.
